(19) United States
(12) Patent Application Publication
(10) Pub. No.: US 2007/0184010 A1
(43) Pub. Date: Aug. 9, 2007

(54) Shaving gel combination

(76) Inventor: Marc S. Herlands, Encinitas, CA (US)

Correspondence Address: Wesley B. Ames;Ames IP Law, 7031 Los Vientos Serenos, Escondido, CA, 92029, US

(21) Appl. No.: 11/346,887

(22) Filed: Feb. 3, 2006

(51) Int. Cl. A61K 8/36

(52) U.S. Cl. 424/73

(57) ABSTRACT

A shaving composition is described that combines a shaving gel, foam, or cream, and a liquid hand or bath soap.

### RELATED APPLICATIONS

NOT APPLICABLE.

### FIELD OF THE INVENTION

The present invention relates to shaving products, and especially to shaving softening and lubricant compositions.

### BACKGROUND OF THE INVENTION

The following discussion is provided solely to assist the understanding of the reader, and does not constitute an admission that any of the information discussed or references cited constitute prior art to the present invention.

A large variety of different compositions have been used for shaving purposes, ranging from solid shaving soaps that can generate a lather, to shaving creams, shaving foams, and to post-foaming shaving gels.

### SUMMARY OF THE INVENTION

This invention concerns advantageous compositions for shaving. Applicants has found that adding a substantial proportion of a liquid hand or bath soap to a shaving gel provides improved lubrication and significantly longer blade life.

Thus, the invention concerns a composition that combines a shaving gel, foam, or cream and a liquid hand or bath soap.

Thus, in a first aspect, the invention provides a shaving composition that includes 20-80% by weight of a shaving gel (such as a post-foaming shaving gel), foam, or cream, and 20-80% by weight of a liquid hand or bath soap.

In particular embodiments the shaving composition includes 40-60% of the shaving gel, foam, or cream, and 40-60% of the liquid hand soap, or 45-55% of the shaving gel, foam, or cream, and 45-55% of the liquid hand soap.

The invention also concerns a shaving composition dispenser that contains a shaving gel, foam, or cream and a liquid soap in separate chambers, such that the dispenser will jointly dispense the shaving gel, foam, or cream and the liquid soap. Thus, another aspect provides a shaving composition dispenser that includes two chambers, where the first chamber contains a shaving gel, foam, or cream, the second chamber contains a liquid hand soap, and the dispenser dispenses a pre-selected ratio of each of the liquid hand soap and the shaving gel, foam, or cream.

In particular embodiments, the shaving gel, foam, or cream and the soap are dispensed from pressurized chambers; the shaving gel, foam, or cream and the soap are dispensed from a common aperture; the shaving gel, foam, or cream and the soap are dispensed from separate apertures.

A related aspect concerns a shaving composition dispenser that includes two linked containers, one of which contains a shaving gel, shaving foam, or shaving cream, and the other of which contains a liquid soap, typically a liquid hand or bath soap. Such linkage can, for example, involve a single outer container wall with a single internal barrier wall, separate containers that are joined by welding, or separate containers that are joined with a plastic or metal band. Each container has a separate actuator, usually but not necessarily leading to a separate outlet.

In yet another aspect, the invention concerns a shaving method that involves applying a combination of a shaving gel, foam, or cream and a liquid hand or bath soap to a skin surface to be shaved, and shaving that skin surface. For example, the method can include applying a combination of one part by weight of a shaving gel, foam, or cream and 0.2-5.0 parts by weight of a liquid hand soap to a skin surface to be shaved, and shaving that skin surface.

In particular embodiments, the combination is as described for an above aspect.

Additional embodiments will be apparent from the Detailed Description and from the claims.

### BRIEF DESCRIPTION OF THE DRAWINGS

FIG. 1 shows an exemplary dual dispenser for a shaving gel, foam, or cream and liquid soap combination.

FIG. 2 shows an exemplary dual dispenser for a shaving gel, foam, or cream and liquid hand soap combination that has two separate chambers and separate actuators and dispensing apertures.

### DETAILED DESCRIPTION OF THE PREFERRED EMBODIMENTS

The present invention concerns advantageous combinations of shaving gel (or shaving foam or cream) and hand or bath soap. The combination remains gentle on the skin while providing high levels of lubrication and extended shaving blade life.

In certain embodiments, the combination is provided as separate compositions that are jointly dispensed and mixed for use. Such combinations have the advantage that the foaming properties of the shaving gel or foam are not substantially impaired.

In other embodiments, a new formulation is provided that includes a substantially higher proportion of soap or detergent components than conventional shaving gels. Thus, the formulation can be regarded as a mixture of a shaving gel and a liquid hand or bath soap.

In many cases, shaving gels contain a combination of water, soap, gelling component(s), and post-foaming gas, along with additional components such as fragrances, emollients, and/or glidants.

A number of shaving gels, especially post-foaming gels, that can be used have been described. For example, U.S. Pat. Nos. 6,916,468; 6,878,754, 6,353,689; 5,902,778; 5,902,574; 5,858,343; 5,759,531; 5,560,859; 5,523,081; 5,262,154; 5,248,495; 5,034,220; 4,871,530; 4,528,111; 3,541,581, as well as patent application publications 2004/0166086; 2004/0166085; and 2003/0170198 describe such shaving gels.

Likewise a large number of different liquid hand and bath soaps can be used. In many cases, such liquid soaps contain a soap or detergent, along with additional ingredients conventional for such liquid hand soaps.

Examples of liquid hand and bath soaps that can be used have been described. For example, MacGilp et al. U.S. Pat. No. 5,296,158; Shields U.S. Pat. No. 4,617,148; Holsopple U.S. Pat. No. 4,556,510; Beattie U.S. Pat. No. 4,430,245; Matsuda U.S. Pat. No. 4,312,771; Brouwer U.S. Pat. No. 4,310,432 describe such liquid hand and bath soaps.

The present shaving gel/hand soap combinations can be dispensed from a number of different types of containers, e.g., pressurized or pump containers as currently used for shaving gels and/or liquid hand soaps. In the case of combination formulations (i.e., shaving formulations containing components similar to the combined ingredients of current shaving gels and liquid hand soaps, a single chamber dispenser can be used, such as the pressurized dispenser cans typically used for dispensing post-foaming shaving gels. Such dispenser cans commonly incorporate a gas-pressurized bag-in-can cr diaphragm design, or a spring-pressurized divider. Such dispensers can include valve assemblies as described and/or commonly used for use with post-foaming gels or with aerosol gels.

The stable post-foaming gel can be packaged in a variety of container systems, preferably containers that include propelling structures such as: (1) mechanically pressurized bag-in-sleeve systems in which a thin-walled inner bag containing the product is surrounded by an outer elastic sleeve that is expanded during the product filling process and provides dispensing power to expel the product (e.g., the ATMOS System available commercially from the Exxel Container Co.); (2) manually activated air pump spray devices in which a pump system is integrated into the container to allow the user to pressurize the container with air in order to expel the product (e.g., the “AIRSPRAY” system available from Airspray International); (3) piston barrier systems (or can with piston) in which the product is separated from the driving means by a tight-fitting piston which seals to the side of the container and may be driven by a spring under tension, by a vacuum on the product side of the piston, by finger pressure, by gas pressure to the piston, or by a variety of other means known to the packaging industry; and (4) bag-in-can (SEPRO) systems in which the product is contained in a flexible bag within a can, with a suitable propellant injected into the space between the can and the flexible bag.

In particular, the “bag-in-can system” basically consists of a metal 3 piece can equipped with an inner plastic bag fastened to the dome that contains the gel/post-foaming agent mixture. The propellant is loaded through the bottom of the can which is perforated for a charging plug, and the propellant remains separate from the bag. The container is designed to use hydrocarbons, and other compressed gasses. The bag serves as a barrier to prevent the gel/post-foaming agent mixture from coming in contact with the propellant around the bag. The bag is made of polyethylene, Surlyn and nylon and has pleated side walls which enable the bag to collapse upward as the propellant force empties the container. The bag is capable of discharging very viscous materials. A major problem with this unit is gas permeation through the bag into the gel. Permeation occurs “when significant numbers of molecules pass through a barrier during a fixed time period.” Larger molecules permeate more slowly than smaller ones. Entrapment of air in the gel when filling into the can will be minimized by use of a spin filler, which rotates the cans while gel is transferred into the bag.

In addition, manually activated pumps can be used. Manually activated pumps typically include a squeeze trigger or finger-type pumping device as part of the propelling structure. Such pumping devices are well known in the art and typically include a pump chamber, an injection pipe member connected to a vertical pipe portion (piston), a cylinder, and a discharge valve, which communicates with the pump actuator. Fluid (or gel) is forced from the pumping device under pumping pressure generated by the pumping action of an injection pipe member lifted and lowered vertically in a cylinder by an operating mechanism. The injection pipe member includes a vertical pipe portion, corresponding to a piston, which is forced into the cylinder. The fluid pressurized in the pump chamber exerts pressure, through means of a fluid passage in the injection pipe member, onto and through a flexible discharge valve, thereby causing the fluid to be ejected from the actuator. A wide variety of manually activated pump dispensers may be used.

In other cases, the dispenser includes separate chambers for the shaving gel component and the liquid hand soap component. In such dispensers, dispensing of each component may be controlled in various ways, and the dispensing channels may be combined or separate. Thus, actuation of the dispenser may involve separate actuation of dispenser controls for each component. In such configurations, each component will typically, but not necessarily, issue from a separate aperture. Such separate apertures may be completely separate or may be twinned. Alternatively, the separate components may utilize the same aperture. In other embodiments, a single control actuates dispensing of both components jointly; the separate components may combine internally and issue through a common aperture, or may issue through separate apertures, e.g., twinned apertures.

Each of the separate chambers may be configured with propelling structures, such as those described above. In some cases it may be preferred to use a positive displacement mechanism such as a piston in can system in conjunction with separate flexible bags within a single container. The bags ensure that the components are maintained separately, while the piston ensures that a volume (e.g., an aliquot) of each component is dispensed. This will prevent one component (e.g., a less viscous component) from being dispensed in preference to the other component. It is generally preferable that the components be dispensed in a substantially consistent ratio (e.g., within a factor of two or even 1.7, 1.5, 1.4, 1.3, or 1.2); thus, the dispensing mechanism be configured to provide such substantially consistent ratio.

The combination of shaving gel, foam, or cream, and liquid hand or bath soap can also be provided in separate containers that are linked or packaged together. In such cases, dispensing the two items will generally occur through separate valve mechanisms and dispensing orifices. Such linkage or packaging can, for example, involve binding of the two container together with a plastic or metal band(s), gluing together of the containers with or without a pack or frame, or the containers may fit within openings in a pack or frame that holds the containers without the requirement for glue or other adhesive.

Dispensers can be used that will dispense either a single aliquot (or an aliquot of each component) on each actuation, or will dispense essentially continuously while an actuator is held in an actuating position. Valve assemblies of both types are well known and can be used for the present dispensers.

### EXAMPLES

As indicated above, combinations of shaving gel and liquid hand or bath soap that can be used in the present invention are numerous. To illustrate the invention further, an exemplary dual chamber dispenser and an exemplary combination of shaving gel and liquid hand soap are described in the examples below.

### Example 1

### Dual Chamber Dispenser

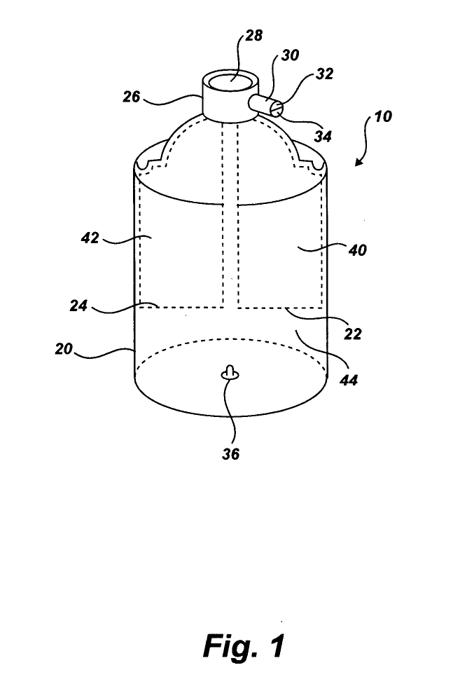

An exemplary combination dispenser is described with reference to FIG. 1. FIG. 1 shows a dual chamber container utilizing a dual bag-in-can design. The dispenser 10 includes the container 20, with valve cap 26. Within the container are two collapsible bags 22 and 24 respectively that contain volumes of shaving gel 40 and liquid hand soap 42. Below the bags is propellant space 44. Propellant is injected into container 20 through bottom, propellant fill port 36, and provides pressure to push the gel and soap out of the container when release valves are actuated. Valve cap 26 includes two internal valve assemblies (not shown) that are controlled by a common trigger 28 releasing both the shaving gel and liquid hand soap through dispensing port 30 through separate channels and apertures 32 and 34. The dispensed combination can then be mixed in the user's hands and applied to the beard area or other skin surface to be shaved.

### Example 2

### Dual Container Dispenser

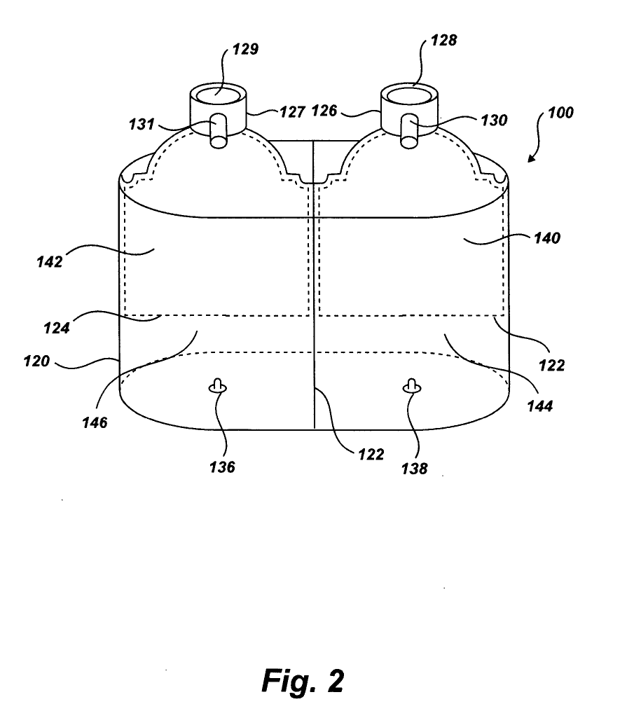

An exemplary dual container dispenser is described with reference to FIG. 2. The dispenser 100 utilizes a bag-in-can design for each chamber. The dispenser includes outer container wall 120 and inner container barrier wall 121 defining 2 separate chambers. Within the chambers are collapsible bags 122 and 124 that contain shaving gel and liquid hand soap respectively. Below the collapsible bags are propellant spaces 144 and 146. Propellant is injected into the propellant spaces through propellant fill ports 136 and 138. Dispensing of the shaving gel and liquid hand soap is controlled using valve caps 126 and 127, which include internal valve assemblies (not shown), valve trigger caps 128 and 129, and outlet spouts 130 and 131.

### Example 3

### Exemplary Shaving Gel and Liquid Hand Soap

An exemplary combination of shaving gel and liquid hand soap is described be reference to previously described shaving gel and liquid had soap formulations.

The shaving gel formulation as described in U.S. Pat. No. 5,560,859 (incorporated herein by reference in its entirety) is prepared from the following ingredients as described below:

| Ingredients | % by wt. |
|---|---|
| Water | 77.95 |
| Palmitic Acid (Hystrene 9016) | 9.74 |
| Triethanolamine | 5.65 |
| Propylene Glycol Isostearate (Emerest 2384) | 1.70 |
| Sorbitol (Liponic 70 NC) | 1.46 |
| PEG-14M (Polyox WSRN-3000) | 0.49 |
| Fragrance | 0.39 |
| Hydroxypropylcellulose (Klucel) | 0.02 |
| Dyes (e.g., FD&C Blue #1 and D&C Yellow #10) | Q.S. |
| Isopentane/Isobutane 85/15 Blend | 2.60 |
| | 100.00% |

The palmitic acid was melted at 75.degree.-80.degree. C. The hydroxypropylcellulose and propylene glycol isostearate were mixed to obtain Mixture 1 which was added to the melted palmitic acid. The Polyox WSRN-3000 was dissolved in substantially all the water to obtain Solution 1. Solution 1 was then warmed to 75.degree.-80 degree C. and maintained at this temperature. The sorbitol and dyes were then added to Solution 1 to form Solution 2. Solution 2 was then added to Mixture 1 to obtain Mixture 2. The 50:50 solution of triethanolamine and water was added to Mixture 2 at 75-80 degree C. to obtain Mixture 3. Mixture 3 was then cooled to 50 degree C. and the fragrance was added to obtain Mixture 4. Mixture 4 was then cooled to 2 degree to 8 degree C. and an 85:15 blend of isopentane/isobutane at a 2.60% level was introduced into Mixture 4.

A liquid hand soap for this exemplary combination is described in U.S. Pat. No. 5,296,158 (incorporated herein by reference in its entirety), prepared as described therein. Additional suitable formulations are also described therein.

The Brookfield viscosity of the following liquid soap is about 30,000 cps. The Iodine Value of the fatty acids therein is about zero and its titer is about 59 degree C. This soap has totals of about 10.2% soap and 6.85% free fatty acid and 2.4% sarcosinate. The soap to free fatty acid (FFA) ratio is about 1:0.67.

| | 1A | 1B |
|---|---|---|
| Formula Ingredients | Wt. % | Wt. % |
| Stearic Acid | 7.55 | 4.53 |
| Palmitic Acid | 6.23 | 3.74 |
| Myristic Acid | 8.72 | 5.23 |
| Lauric Acid | 3.52 | 2.11 |
| Triclosan | 0.30 | 0.18 |
| KOH (87%) | 3.86 | 2.32 |
| Glycerine | 15.00 | 9.00 |
| Mayoquest (45%)* | 0.44 | 0.26 |
| Sodium Lauroyl Sarcosinate (30%) | 13.33 | 8.00 |
| JR-400 | 0.50 | 0.30 |
| Aloe Vera Powder | 0.01 | 0.01 |
| Perfume | 0.30 | 0.18 |
| Total Water (approx.) | 50.00 | 70.00 |

*Mayoquest is a 50/50 mixture of HEDP/DPTA

A liquid soap (Formulation 1B) is made by first mixing the ingredients of “1A” as follows:

1. Mix and melt all of the fatty acids with the Triclosan into a jacketed vessel and heat to 80 degree C.

2. Dissolve the KOH pellets with water to make a 38% solution by weight.

3. Mix the glycerine, sodium or potassium lauroyl sarosinate, JR-400, Mayoquest, and water in a separate jacketed vessel and heat to 80 degree C.

4. Transfer the melted fatty acid mix of Step 1 into a vacuum vessel which contains an internal homogenizer, wall scrapers and paddle mixers, e.g., a Mizuho Brand Automatic Driving Type Vacuum Emulsifier, Model APVQ-3DP, sold by Mizuho Industrial Co., Ltd., or a T.K. AGI Homo Mixer Hodel 2M-2, made by Tokushu Kika Kogyo Co., Ltd. While vacuum is not essential, it is highly preferred so that the intermediate product has a specific gravity of about 1±0.05.

5. Slowly add the KOH solution under vacuum of about 400 mm Hg while mixing and homogenizing during saponifying. Maintain temperature controlled to 80±5 degree C. while mixing.

6. After the saponification is complete, add the water mix of Step 3 under vacuum while continuing mixing and homogenizing. Maintain temperature controlled to 80±5 degree C. while mixing to obtain a phase stable melt.

7. Immediately begin cooling from 80 degree C. to 50 degree C. at a 3 degree C./minute rate. Maintain mixing and vacuum during cooling step but stop homogenizing.

8. Dissolve the aloe vera powder in water and add at 50 degree C.

9. Cool from 50 degree C. to 35 degree C. at a 0.5 degree C./minute rate under vacuum and while mixing.

10. At 35 degree C. stop the vacuum and add the perfume. Continue cooling with mixing until final mix reaches about 30 degree C. At 30 degree C., stop cooling and unload the mix from the vessel.

11. The cooled melt of Step 10 (1A) is then diluted with distilled water at about room temperature. The water and the cooled melt is first mixed gently to provide a uniform slurry and then transferred to the vacuum vessel of Step 4 and homogenized for about 10 minutes under about 600 Mm Hg to provide an aqueous (70% water) liquid soap dispersoidal (Formulation 1B).

Formulation 1B can further include stabilizers, such as the combination described in the above-cited patent.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to the shaving gel and liquid hand soap formulations. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values or value range endpoints are provided for embodiments, additional embodiments are described by taking any 2 different values as the endpoints of a range or by taking two different range endpoints from specified ranges as the endpoints of an additional range. Such ranges are also within the scope of the described invention.

Thus, additional embodiments are within the scope of the invention and within the following claims.

What is claimed is:

1. A shaving composition, comprising
20-80% by weight of a shaving gel, shaving foam, or shaving cream; and
20-80% by weight of a liquid hand soap.

2. The shaving composition of claim 1, comprising 40-60% by weight of said shaving gel, and 40-60% by weight of said liquid hand soap.

3. The shaving composition of claim 1, wherein said shaving gel is a post-foaming shaving gel.

4. The shaving composition of claim 1, wherein said shaving gel, shaving foam, or shaving cream is an aerosol foam.

5. The shaving composition of claim 1, wherein said shaving gel, shaving foam, or shaving cream is a shaving cream.

6. A shaving composition dispenser comprising
two chambers, wherein the first chamber contains a shaving gel, shaving foam, or shaving cream, the second chamber contains a liquid hand soap, and said dispenser dispenses a pre-selected ratio of each of said liquid and said shaving gel, shaving foam, or shaving cream.

7. The dispenser of claim 6, wherein said shaving gel, foam, or cream, and said soap are dispensed from pressurized chambers.

8. The dispenser of claim 6, wherein said shaving gel, foam, or cream, and said soap are dispensed from a common aperture.

9. The dispenser of claim 6, wherein said shaving gel, foam, or cream, and said soap are dispensed from separate apertures.

10. The dispenser of claim 6, wherein said shaving gel, foam, or cream is a post-foaming shaving gel.

11. The dispenser of claim 6, wherein said shaving gel, foam, or cream is an aerosol foam.

12. The dispenser of claim 6, wherein said shaving gel, foam, or cream is a shaving cream.

13. A method for shaving hair from a skin surface, comprising
applying a combination of one part by weight of a shaving gel and 0.2-5.0 parts by weight of a liquid hand soap to a skin surface to be shaved; and
shaving said skin surface.
